(19) United States
(12) Patent Application Publication
(10) Pub. No.: US 2012/0171683 A1
(43) Pub. Date: Jul. 5, 2012

(54) ANALYSIS OF FRAGMENTED GENOMIC DNA IN DROPLETS

(76) Inventors: Kevin D. Ness, Pleasanton, CA (US); Amy L. Hiddessen, Dublin, CA (US); Paul W. Wyatt, Pleasanton, CA (US)

(21) Appl. No.: 13/287,095

(22) Filed: Nov. 1, 2011

Related U.S. Application Data
(63) Continuation-in-part of application No. 12/976,827, filed on Dec. 22, 2010.
(60) Provisional application No. 61/309,845, filed on Mar. 2, 2010.
(60) Provisional application No. 61/341,218, filed on Mar. 25, 2010.
(60) Provisional application No. 61/317,635, filed on Mar. 25, 2010.
(60) Provisional application No. 61/380,981, filed on Sep. 8, 2010.
(60) Provisional application No. 61/409,106, filed on Nov. 1, 2010.
(60) Provisional application No. 61/409,473, filed on Nov. 2, 2010.
(60) Provisional application No. 61/410,769, filed on Nov. 5, 2010.
(60) Provisional application No. 61/417,241, filed on Nov. 25, 2010.

(51) Int. Cl. C12Q 1/68

(52) U.S. Cl. 435/6.12; 435/6.1

(57) ABSTRACT

Method of analyzing genomic DNA. Genomic DNA including a target may be obtained. The genomic DNA may be fragmented volitionally to produce fragmented DNA. The fragmented DNA may be passed through a droplet generator to generate aqueous droplets containing the fragmented DNA. An assay may be performed on the droplets to determine a level of the target. In some embodiments, the droplets may contain the genomic DNA at a concentration of at least about five nanograms per microliter, the droplets may be generated at a droplet generation frequency of at least about 50 droplets per second, the droplets may have an average volume of less than about 10 nanoliters per droplet, the droplets may generated at a flow rate of greater than about 50 nanoliters per second, or any combination thereof.

### CROSS-REFERENCES TO PRIORITY APPLICATIONS

This application is a continuation-in-part of U.S. patent application Ser. No. 12/976,827, filed Dec. 22, 2010, published as U.S. Patent Application Publication No. 2011/0217712 A1 on Sep. 8, 2011, which, in turn, is based upon and claims the benefit under 35 U.S.C. §119(e) of the following U.S. provisional patent applications: Ser. No. 61/309,845, filed Mar. 2, 2010; Ser. No. 61/341,218, filed Mar. 25, 2010; Ser. No. 61/317,635, filed Mar. 25, 2010; Ser. No. 61/380,981, filed Sep. 8, 2010; Ser. No. 61/409,106, filed Nov. 1, 2010; Ser. No. 61/409,473, filed Nov. 2, 2010; Ser. No. 61/410,769, filed Nov. 5, 2010; and Ser. No. 61/417,241, filed Nov. 25, 2010.

Each of these patent applications is incorporated herein by reference in its entirety for all purposes.

### CROSS-REFERENCES TO ADDITIONAL MATERIALS

This application incorporates herein by reference in their entirety for all purposes the following materials: U.S. Pat. No. 7,041,481, issued May 9, 2006; U.S. Patent Application Publication No. 2010/0173394 A1, published Jul. 8, 2010; PCT Patent Application No. WO 2011/120024, published Sep. 29, 2011; and Joseph R. Lakowicz, PRINCIPLES OF FLUORESCENCE SPECTROSCOPY (2^{nd} Ed. 1999).

### INTRODUCTION

Many biomedical applications rely on high-throughput assays of samples for nucleic acid targets. For example, in research and clinical applications, high-throughput genetic tests using target-specific reagents can provide accurate and precise quantification of nucleic acid targets for drug discovery, biomarker discovery, and clinical diagnostics, among others.

Emulsions hold substantial promise for revolutionizing high-throughput assays for targets. Emulsification techniques can create large numbers of aqueous droplets that function as independent reaction chambers for biochemical reactions. For example, an aqueous sample (e.g., 20 microliters) can be partitioned into droplets (e.g., 20,000 droplets of one nanoliter each) to allow an individual test for the target to be performed with each of the droplets.

Aqueous droplets can be suspended in oil to create a water-in-oil emulsion (W/O). The emulsion can be stabilized with a surfactant to reduce coalescence of droplets during heating, cooling, and transport, thereby enabling thermal cycling to be performed. Accordingly, emulsions have been used to perform single-copy amplification of nucleic acid target molecules in droplets using the polymerase chain reaction (PCR). Digital assays are enabled by the ability to detect the presence of individual molecules of a target in droplets.

In an exemplary droplet-based digital assay, a sample is partitioned into a set of droplets at a limiting dilution of a target (i.e., some of the droplets contain no molecules of the target). If molecules of the target are distributed randomly among the droplets, the probability of finding exactly 0, 1, 2, 3, or more target molecules in a droplet, based on a given average concentration of the target in the droplets, is described by a Poisson distribution. Conversely, the concentration of target molecules in the droplets (and thus in the sample) may be calculated from the probability of finding a given number of molecules in a droplet.

Estimates of the probability of finding no target molecules and of finding one or more target molecules may be measured in the digital assay. In a binary approach, each droplet can be tested to determine whether the droplet is positive and contains at least one molecule of the target, or is negative and contains no molecules of the target. The probability of finding no molecules of the target in a droplet can be approximated by the fraction of droplets tested that are negative (the “negative fraction”), and the probability of finding at least one target molecule by the fraction of droplets tested that are positive (the “positive fraction”). The value of the positive fraction or the negative fraction then may be utilized in a Poisson algorithm to calculate the concentration of the target in the droplets. In other cases, the digital assay may generate data that is greater than binary. For example, the assay may measure how many molecules of the target are present in each droplet with a resolution greater than negative (0) or positive (>0) (e.g., 0, 1, or >1 molecules; 0, 1, 2, or >2 molecules; or the like).

For a combination of high throughput and accuracy in droplet-based DNA assays of different samples, droplets should be generated rapidly and with a uniform size (i.e., monodisperse droplets). However, sample components can interfere with the ability of droplets to separate from the bulk sample phase, particularly as the frequency of droplet generation is increased. As a result, the size of droplets formed, or even the ability to form droplets at all, can vary from sample to sample, diminishing the reliability of the assays. New approaches are needed to provide reliable and consistent generation of droplets at a higher generation frequency.

### SUMMARY

The present disclosure provides a method of analyzing genomic DNA. Genomic DNA including a target may be obtained. The genomic DNA may be fragmented volitionally to produce fragmented DNA. The fragmented DNA may be passed through a droplet generator to generate aqueous droplets containing the fragmented DNA. A digital assay may be performed on the droplets to determine a level of the target. In some embodiments, the droplets may contain the genomic DNA at a concentration of at least about five nanograms per microliter, the droplets may be generated at a droplet generation frequency of at least about 50 droplets per second, the droplets may have an average volume of less than about 10 nanoliters per droplet, the droplets may generated at a sample flow rate of greater than about 50 nanoliters per second, or any combination thereof.

### BRIEF DESCRIPTION OF THE DRAWINGS

FIG. 1 is a flowchart illustrating an exemplary method of analyzing genomic DNA, in accordance with aspects of the present disclosure.

FIG. 2 is a matrix of drawings made from photographs of a droplet generator processing three different samples at each of four different driving pressures.

FIG. 3 is a graph of droplet volume plotted as a function of droplet generation frequency for samples containing no genomic DNA or genomic DNA (Raji or Coriell) that is digested (EcoRI) or undigested.

FIG. 4 is a graph of droplet volume plotted as a function of sample flow rate for the samples of FIG. 3.

FIG. 5 is a graph of maximum extension plotted as a function of droplet generation frequency for the samples of FIG. 3.

FIG. 6 is a graph of maximum extension plotted as a function of sample flow rate for the samples of FIG. 3.

### DETAILED DESCRIPTION

The present disclosure provides a method of analyzing genomic DNA. Genomic DNA including a target may be obtained. The genomic DNA may be fragmented volitionally to produce fragmented DNA. The fragmented DNA may be passed through a droplet generator to generate aqueous droplets containing the fragmented DNA. An assay may be performed on the droplets to determine a level of the target. In some embodiments, the droplets may contain the genomic DNA at a concentration of at least about five nanograms per microliter, the droplets may be generated at a droplet generation frequency of at least about 50 droplets per second, the droplets may have an average volume of less than about 10 nanoliters per droplet, the droplets may generated at a flow rate of greater than about 50 nanoliters per second, or any combination thereof.

The method of analyzing genomic DNA in droplets, as disclosed herein, has substantial advantages over other droplet-based approaches. The advantages may include generating droplets at a higher frequency, with greater monodispersity, with a higher load of DNA, and/or with substantially less interference from genomic DNA.

These and other aspects of the present disclosure are described in the following sections: (I) overview of an exemplary method of genomic DNA analysis, (II) exemplary data from tests of droplet generation, and (III) selected embodiments.

### I. Overview of an Exemplary Method of Genomic DNA Analysis

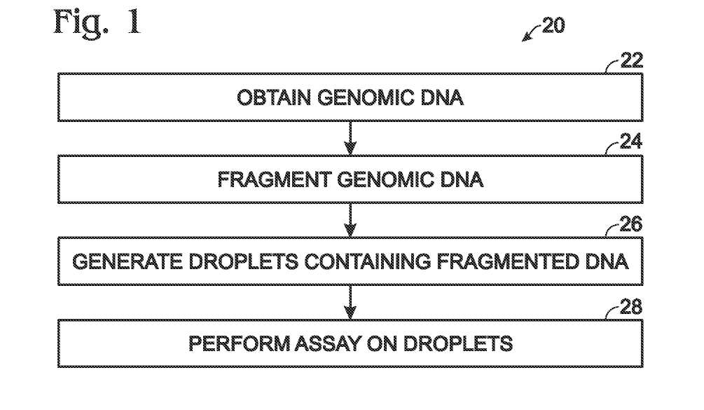

FIG. 1 shows a flowchart illustrating an exemplary method 20 of analyzing genomic DNA. The steps presented may be performed in any suitable order and in any suitable combination.

Genomic DNA may be obtained, indicated at 22. The DNA may be obtained from any suitable organism, such as a mammal (e.g., human, mouse, rat, monkey, etc.), a non-mammalian vertebrate, an invertebrate, a yeast or fungus, a plant, a protozoan, a bacterium, or the like. The DNA may be obtained by any suitable process, such as purchased commercially, received as a gift, acquired by extraction from cells or fluid, received as a clinical sample, or the like. The DNA may be obtained in a relatively high molecular weight form, such as having a molecular weight of at least about 10⁴, 10⁵ or 10⁶ kilodaltons, among others (e.g., having an average length of at least about 25, 50, 100, 200, 500, or 1,000 kilobases).

The genomic DNA may be fragmented, indicated at 24, before droplet generation. Fragmentation may be a volitional act, that is, performed deliberately. Fragmentation generally involves any procedure that substantially reduces the molecular weight of the genomic DNA, such as by cutting or breaking DNA strands. The fragmentation may reduce the average molecular weight and/or length by any suitable amount, such as at least about 5, 10, 20, 50, or 100-fold, among others. An exemplary approach to fragmenting genomic DNA includes digestion with a restriction enzyme (e.g., an enzyme having a 4, 5, 6 or 8 nucleotide recognition site, among others). The target may contain no recognition sites for the restriction enzyme, to avoid any cleavage of target molecules. The restriction enzyme digestion may be performed to completion or may be a partial digestion. Alternatively, or in addition, an aqueous sample of the genomic DNA may be heated to fragment the DNA. Exemplary heating that fragments the DNA may be performed at a temperature of at least 95° C., for at least about 10, 15, 20, or 30 minutes, among others. In other cases, the DNA may be fragmented by shearing, sonicating, nebulizing, irradiating, or the like.

The genomic DNA may include a target, generally a sequence of interest to be tested. Fragmentation of the genomic DNA may be performed without substantially disrupting the target, meaning that less than one-half of target sequences in the genomic DNA are disrupted (e.g., broken or cut) by the fragmentation process. Droplets containing the fragmented DNA may be generated, indicated at 26. The droplets may be generated serially with each of one or more droplet generators. The fragmented DNA may be passed through at least one droplet generator to generate droplets. Generally, the fragmented DNA is disposed in an aqueous sample, and the aqueous sample and an immiscible continuous phase are passed through the droplet generator to form aqueous droplets containing the fragmented DNA and disposed in the continuous phase. Further aspects of droplet generators and emulsion phases that may be suitable are described in the documents listed above under Cross-References, which are incorporated herein by reference, particularly U.S. Patent Application Publication No. 2010/0173394 A1, published Jul. 8, 2010; and PCT Patent Application No. WO 2011/120024, published Sep. 29, 2011.

The droplets may have any suitable size. For example, the droplets may have an average volume of less than about 1 μL, 100 nL, 10 nL, 1 nL, 100 pL, 10 pL, or 1 pL, among others. Alternatively, or in addition, the droplets may have an average volume of greater than about 10 fL, 100 fL, 1 pL, 10 pL, or 100 pL, among others. In some cases, the droplets may have an average volume of about 1 pL to 100 nL, 1 pL to 10 nL, or 0.1 to 10 nL, among others. The droplets may be monodisperse.

The droplets may contain any suitable concentration of fragmented DNA. For example, the fragmented DNA may be disposed in the droplets at a concentration of at least about 0.1, 0.2, 0.5, 1, 2, 5, 10, 20, 50 ng/μL, among others. In some cases, the concentration may be about 0.1-50 or 0.2-20 ng/μL. Fragmenting the DNA allows a higher DNA load to be incorporated into droplets. The fragmented DNA may be present at an average of less than about two genome-equivalents per droplet. The target may be present at an average of less than about two molecules per droplet.

The droplets may be formed at any suitable droplet generation frequency, such as at least about 10, 20, 50, 100, 200, 500, or 1,000 Hz (droplets/second), among others. Generally, the droplet generation frequency is inversely related to the size of droplets being generated, with smaller droplets allowing a higher droplet generation frequency.

An aqueous sample used to form the droplets (and containing the fragmented DNA) may be passed through the droplet generator and/or converted into droplets at any suitable flow rate. Exemplary flow rates that may be suitable include at least about 1, 5, 10, 20, 50, 100, 200, 500, 1,000, 5,000, or 10,000 nL/second, among others. Generally, the sample flow rate is directly related to the size of droplets being generated, with larger droplets permitting a higher flow rate.

The exemplary values (or ranges) for droplet volumes, DNA concentrations, droplet generation frequencies, and flow rates listed above may be combined in any suitable combination(s).

An assay may be performed on the droplets, indicated at 28. The assay may be a digital assay that detects individual target molecules in the droplets. The digital assay may involve amplifying target molecules, such as by PCR or a ligase chain reaction, among others. The digital assay also may involve detecting fluorescence from the droplets. The assay further may involve determining a level (e.g., a concentration) of the target in the droplets with a Poisson algorithm.

II. Exemplary Data from Tests of Droplet Generation

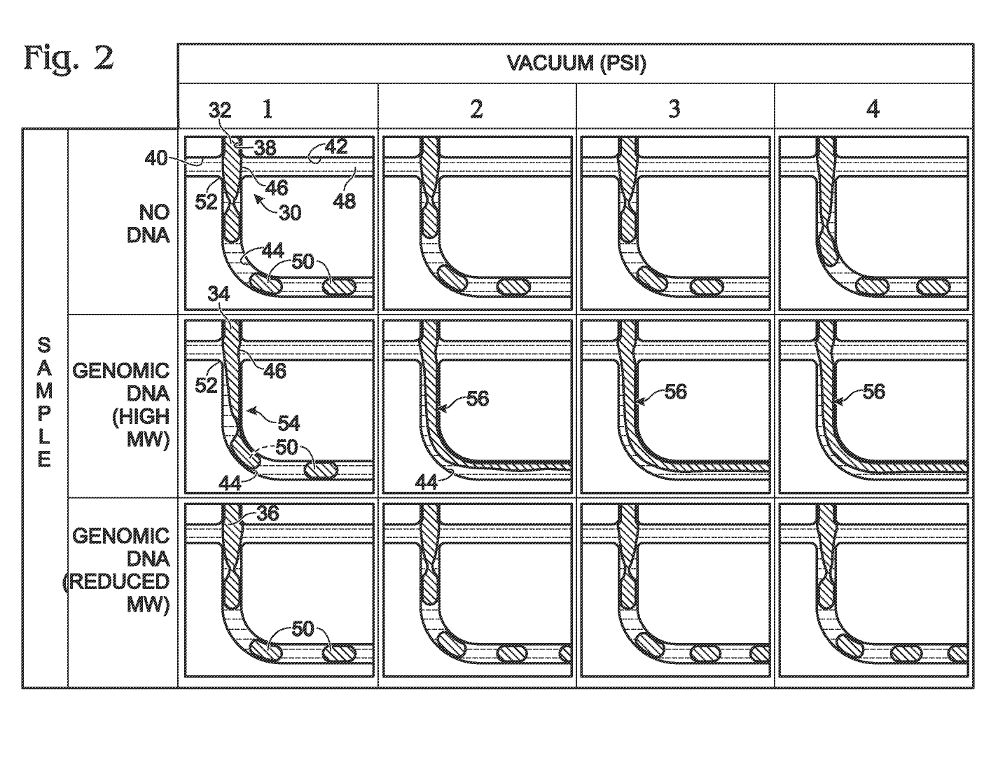

This section present exemplary data from tests of droplet generation with genomic DNA, with or without fragmentation; see FIGS. 2-6.

Droplet generation in a microfluidic device may depend on the flow rate at which the sample travels to a droplet generator, and on the frequency with which droplets are generated. At high flow rates, a sample stream may jet into the immiscible continuous phase, no longer generating droplets. At generation rates close to the jetting limit, the sample starts to extend deeper into the outlet channel before droplets are generated. This extension length can be used to see how close a set of generation conditions is to the jetting limit.

FIG. 2 shows a matrix of drawings made from photographs of a droplet generator 30 processing three different aqueous samples 32-36 at each of four different driving pressures and thus flow rates. The three samples are (a) a control sample 32 containing no DNA (PCR buffer with no template), (b) an aqueous sample 34 of human genomic DNA (Raji, 18.75 ng/μL) that is undigested and has a high molecular weight (MW), and (c) an aqueous sample 36 of human genomic DNA (Raji, 18.75 ng/μL) that has been digested with a restriction enzyme and has a reduced molecular weight. The four driving pressures (1, 2, 3, and 4) are negative (vacuum) pressures applied downstream of the droplet generator and are expressed in pounds per square inch (psi), with 1 psi equal to about 6.9 kilopascals. The vacuum level may control the sample flow rate, the total flow rate, and the droplet generation frequency.

Droplet generator 30 may be formed by a channel network composed of a sample inlet channel 38, at least one or a pair of oil inlet channels 40, 42, and an outlet channel 44. Inlet channel 38 carries a bulk aqueous phase 46 of aqueous sample 32, 34, or 36 to the droplet generator. Inlet channels 40, 42 carry a continuous phase 48 (e.g., oil with a surfactant) to the droplet generator. Outlet channel 44 carries droplets 50 in continuous phase 48 away from a channel intersection 52.

The top row shows droplet generation of control sample 32, which contains no genomic DNA. Droplets 50 are approximately 1 nL and do not vary much in size with the different vacuum levels.

The middle row shows droplet generation with sample 34 containing undigested genomic DNA. The genomic DNA strongly impairs droplet generation, which occurs only at the lowest vacuum level tested (1 psi). Even at this lowest level, there is a considerable extension of bulk aqueous phase 46 past channel intersection 52, indicated by an arrow at 54, and droplets 50 are larger. At higher vacuum levels (e.g., compare 1 psi with 2-4 psi) and flow rates, no droplets are generated, because the sample stream jets into outlet channel 44, indicated by an arrow at 56, without breaking up into droplets. Accordingly, the presence of human genomic DNA can strongly interfere with droplet generation, and may require use of lower DNA concentrations, flow rates, and droplet generation frequencies. As a consequence, sample processing may be slowed considerably. Also, the frequency of target-positive droplets in the emulsion may be reduced substantially (due to the lower DNA concentration), which would require more droplets to be analyzed to achieve to the same confidence for the target level determined.

The bottom row shows droplet generation with sample 36 containing the same concentration (mass per unit volume) of genomic DNA as sample 34, but after the DNA has been digested with a restriction enzyme into shorter fragments. At the pressures (and flow rates) shown here, the genomic DNA in fragmented form does not detectably impair droplet generation. The sample produces droplets 50 that are similar to control sample 32 lacking DNA.

Further studies were conducted to quantitatively measure relationships among the generation vacuum, sample flow rate, droplet generation frequency, velocity in the outlet channel, droplet size, and maximum sample extension during droplet generation. The aqueous samples used were Spectral Dye Buffer (the same control sample as in FIG. 2, but without DNA polymerase), Raji human genomic DNA (Loftstrand Laboratories) (“Raji”), and 19205 human DNA (Coriell Institute) (“Corell”). DNA samples were either undigested or digested with a restriction enzyme, EcoRI. DNA digestion was performed with a 20 U/μL concentration of EcoRI (New England Biolabs) in NEB #4 buffer, with the genomic DNA at a final concentration of 200 ng/μL. The mixture was incubated at 37° C. for one hour, and then was diluted to various final concentrations.

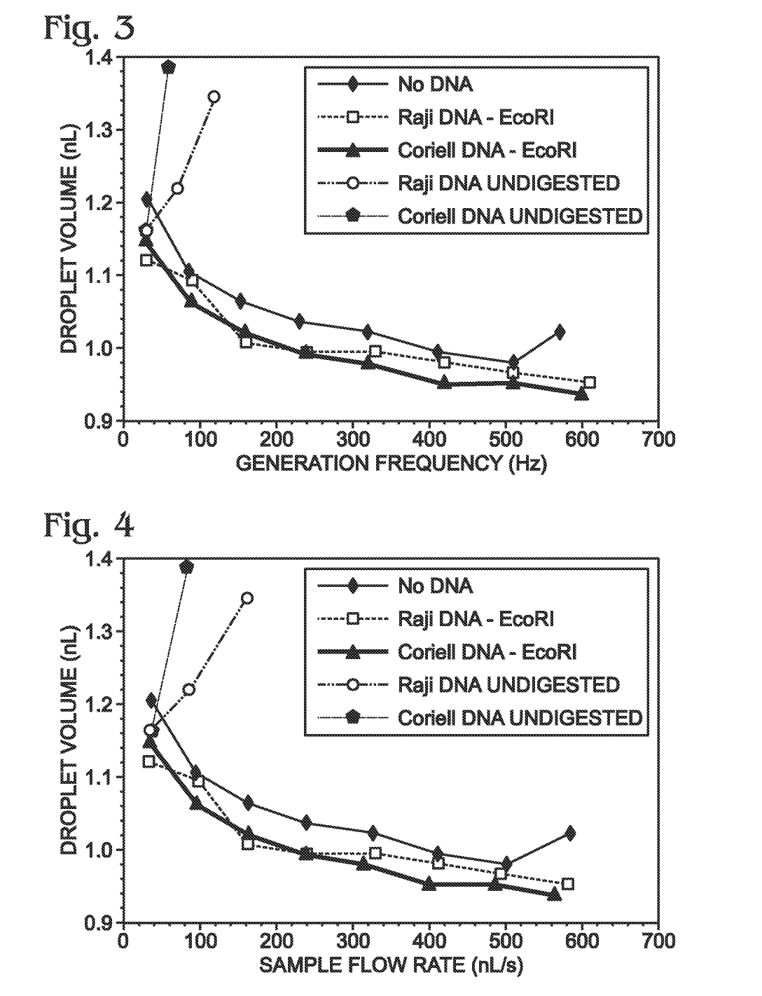

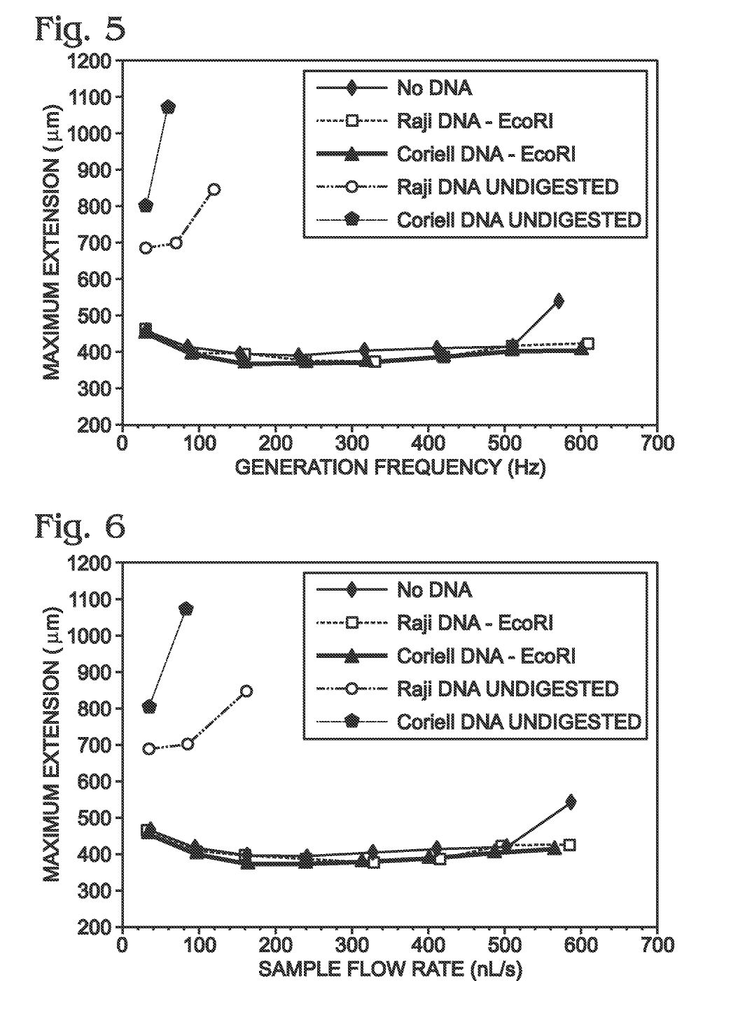

The graphs of FIGS. 3-6 show the results of droplet generation experiments performed with samples of Master Mix (no DNA), EcoRI-digested Raji DNA at 18.75 ng/μL, EcoRI-digested Coriell 19205 DNA at 18.75 ng/μL, undigested Raji DNA at 18.75 ng/μL, and undigested Coriell 19205 DNA at 18.75 ng/μL. FIG. 3 shows a graph of droplet volume plotted as a function of droplet generation frequency for the samples. FIG. 4 shows a graph of droplet volume plotted as a function of sample flow rate for the samples. FIG. 5 shows a graph of maximum extension plotted as a function of droplet generation frequency for the samples. FIG. 6 shows a graph of maximum extension plotted as a function of sample flow rate for the samples of FIG. 3.

For the undigested human genomic DNA, only very low droplet generation frequencies or sample flow rates were possible before jetting occurred. For instance, for Coriell 19205 DNA, the maximum was 60 Hz, and 83 nL/sec. For Raji DNA, the maximum was 120 Hz, and 162 nL/sec. However, even below these limits the generated droplets had a higher volume than in the absence of DNA, and generation occurred with much longer sample extension into the output channel.

The graphs also show that for digested DNA at the same concentration, the effects of DNA on droplet generation are not detectable. No jetting or long sample extensions were observed at any of the flow rates or generation frequencies that were tested, and the droplet volumes are the same as with the sample with no DNA present. These results show that droplet generation is strongly impaired in the presence of undigested human DNA, but not after digestion with a restriction enzyme.

### III. SELECTED EMBODIMENTS

This section describes selected embodiments of the present disclosure as a series of indexed paragraphs. These embodiments should not limit the entire scope of the present disclosure.

A. A method of analyzing genomic DNA, comprising: (i) obtaining genomic DNA including a target; (ii) fragmenting the genomic DNA volitionally to produce fragmented DNA; (iii) passing the fragmented DNA through at least one droplet generator to generate aqueous droplets containing the fragmented DNA; and (iv) performing a digital assay on the droplets to determine a level of the target.

B. The method of paragraph A, wherein the droplets have an average volume of less than about 10 nanoliters.

C. The method of paragraph A, wherein the droplets contain the genomic DNA at a concentration of at least about 5 nanograms per microliter.

D. The method of paragraph A, wherein the genomic DNA is disposed in an aqueous sample, and wherein the droplets are generated at a flow rate of greater than about 50 nanoliters per second of the aqueous sample through the droplet generator.

E. The method of any of paragraphs A to D, wherein the droplets are generated at a droplet generation frequency of at least about 50 droplets per second.

F. The method of paragraph A, wherein the droplets have an average volume of less than about 10 nanoliters and contain the genomic DNA at a concentration of at least about 5 nanograms per microliter.

G. The method of paragraph A, wherein the droplets have an average volume of less than about 10 nanoliters, wherein the genomic DNA is disposed in an aqueous sample, and wherein the droplets are generated at a flow rate of greater than about 50 nanoliters per second of the aqueous sample through the droplet generator.

H. The method of paragraph A, wherein the droplets contain the genomic DNA at a concentration of at least about 5 nanograms per microliter, wherein the genomic DNA is disposed in an aqueous sample, and wherein the droplets are generated at a flow rate of greater than about 50 nanoliters per second of the aqueous sample through the droplet generator.

I. The method of paragraph F, wherein the genomic DNA is disposed in an aqueous sample, and wherein the droplets are generated at a flow rate of greater than about 50 nanoliters per second of the aqueous sample through the droplet generator.

J. The method of paragraph F, wherein the droplets are generated at a droplet generation frequency of at least about 50 droplets per second.

K. The method of paragraph G, wherein the droplets are generated at a droplet generation frequency of at least about 50 droplets per second.

L. The method of paragraph H, wherein the droplets are generated at a droplet generation frequency of at least about 50 droplets per second.

M. The method of paragraph L, wherein the droplets have an average volume of less than about 10 nanoliters.

N. The method of any of paragraphs A to M, wherein the step of fragmenting includes a step of digesting the genomic DNA with a restriction enzyme.

O. The method of paragraph N, wherein the restriction enzyme cuts the genomic DNA an average of less than about once every kilobase.

P. The method of any of paragraphs A to M, wherein the step of fragmenting includes a step of shearing the genomic DNA.

Q. The method of any of paragraphs A to M, wherein the step of fragmenting includes a step of sonicating the genomic DNA.

R. The method of any of paragraphs A to Q, wherein the droplets contain an average of less than about two copies of the target per droplet.

S. The method of any of paragraphs A to R, wherein the droplets contain an average of less than about two genome-equivalents of the genomic DNA per droplet.

T. The method of any of paragraphs A to S, wherein the step of fragmenting does not disrupt the target substantially.

U. The method of any of paragraphs A to T, wherein the step of performing a digital assay includes a step of amplifying the target in the droplets.

V. The method of paragraph U, wherein the target is amplified by PCR.

W. The method of any of paragraphs A to V, wherein the step of performing a digital assay includes a step of detecting fluorescence of the droplets.

X. The method of any of paragraphs A to W, wherein the step of performing a digital assay includes a step of determining a level of the target with a Poisson algorithm.

Y. The method of any of paragraphs A to X, wherein the droplets have an average volume of about 0.1 to 10 nanoliters.

Z. A method of partitioning an aqueous sample comprising DNA into droplets, the method comprising: (i) obtaining a sample comprising DNA at a concentration of at least about 5 ng per microliter; (ii) fragmenting the DNA volitionally to produce fragmented DNA; and (iii) passing the sample through a droplet generator, to generate aqueous droplets containing the fragmented DNA, the droplets being generated at a droplet generation frequency of at least about 50 droplets per second and having an average volume of less than about 10 nanoliters.

A1. A method of partitioning an aqueous sample comprising DNA into droplets, the method comprising: (i) obtaining a sample comprising genomic DNA; (ii) fragmenting the DNA volitionally to produce fragmented DNA; and (iii) passing the sample through a droplet generator, to generate aqueous droplets containing the fragmented DNA, the droplets being generated at a droplet generation frequency of at least about 50 droplets per second and having an average volume of less than about 10 nanoliters, wherein the genomic DNA is at a concentration that interferes with droplet generation if the step of passing is performed with the genomic DNA under the same conditions without fragmenting the DNA.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

1. A method of analyzing genomic DNA, comprising:
obtaining genomic DNA including a target;
fragmenting the genomic DNA volitionally to produce fragmented DNA;
passing the fragmented DNA through a droplet generator, to generate aqueous droplets containing the fragmented DNA at a concentration of at least about 5 nanograms per microliter, the droplets being generated at a droplet generation frequency of at least about 50 droplets per second and having an average volume of less than about 10 nanoliters; and
performing a digital assay on the droplets to determine a level of the target.

2. The method of claim 1, wherein the genomic DNA is disposed in an aqueous sample, and wherein the droplets are generated at a flow rate of greater than about 50 nanoliters per second of the aqueous sample through the droplet generator.

3. The method of claim 1, wherein the droplets have an average volume of about 0.1 to 10 nanoliters.

4. The method of claim 3, wherein the genomic DNA is disposed in an aqueous sample, and wherein the droplets are generated at a flow rate of greater than about 50 nanoliters per second of the aqueous sample through the droplet generator.

5. The method of claim 1, wherein the step of fragmenting includes a step of digesting the genomic DNA with a restriction enzyme.

6. The method of claim 5, wherein the restriction enzyme cuts the genomic DNA an average of less than about once every kilobase.

7. The method of claim 1, wherein the step of fragmenting includes a step of shearing the genomic DNA.

8. The method of claim 1, wherein the step of fragmenting includes a step of sonicating the genomic DNA.

9. The method of claim 1, wherein the droplets contain an average of less than about two copies of the target per droplet.

10. The method of claim 1, wherein the droplets contain an average of less than about two genome-equivalents of the genomic DNA per droplet.

11. The method of claim 1, wherein the step of fragmenting does not disrupt the target substantially.

12. The method of claim 1, wherein the step of performing a digital assay includes a step of amplifying the target in the droplets.

13. The method of claim 12, wherein the target is amplified by PCR.

14. The method of claim 12, wherein the step of performing a digital assay includes a step of detecting fluorescence from the droplets.

15. The method of claim 12, wherein the step of performing a digital assay includes a step of determining a level of the target with a Poisson algorithm.

16. A method of partitioning an aqueous sample comprising DNA into droplets, the method comprising:
obtaining a sample comprising DNA at a concentration of at least about 5 ng per microliter;
fragmenting the DNA volitionally to produce fragmented DNA; and
passing the sample through a droplet generator, to generate aqueous droplets containing the fragmented DNA, the droplets being generated at a droplet generation frequency of at least about 50 droplets per second and having an average volume of less than about 10 nanoliters.

17. A method of partitioning an aqueous sample comprising DNA into droplets, the method comprising:
obtaining a sample comprising genomic DNA;
fragmenting the DNA volitionally to produce fragmented DNA; and
passing the sample through a droplet generator, to generate aqueous droplets containing the fragmented DNA, the droplets being generated at a droplet generation frequency of at least about 50 droplets per second and having an average volume of less than about 10 nanoliters,
wherein the genomic DNA is at a concentration that interferes with droplet generation if the step of passing is performed under the same conditions without fragmenting the DNA.
